(19) United States
(12) Patent Application Publication
(10) Pub. No.: US 2021/0219924 A1
(43) Pub. Date: Jul. 22, 2021

(54) Noninvasive Diagnostics of Proximal Heart Health Biomarkers

(71) Applicants: Alexander S. Liberson, Pittsford, NY (US); Yashar Seyed Vahedein, Ramsey, NJ (US); David A. Borkholder, Canandaigua, NY (US)

(72) Inventors: Alexander S. Liberson, Pittsford, NY (US); Yashar Seyed Vahedein, Ramsey, NJ (US); David A. Borkholder, Canandaigua, NY (US)

(73) Assignee: Rochester Institute of Technology, Rochester, NY (US)

(21) Appl. No.: 17/150,920

(22) Filed: Jan. 15, 2021

Related U.S. Application Data
(60) Provisional application No. 62/961,819, filed on Jan. 16, 2020.

(51) Int. Cl. A61B 5/00; A61B 5/029; A61B 5/02; A61B 5/021; A61B 5/0205; G16H 50/30

(52) U.S. Cl. CPC A61B 5/7278; A61B 5/029; A61B 5/02007; A61B 5/02416; A61B 5/0205; G16H 50/30; A61B 2560/0223; A61B 5/02125

(57) ABSTRACT

An integrated bioinstrumentation system, combining an accurate and robust quasi 1D computational model with experimental peripheral measurements, is designed to extract information on other quantities of interest, for which the direct measurements are not feasible. The system is able to quantify and visualize the distributions of a cardiac output (CO), aortic blood pressure (BP), flow, velocity, and aortic arterial compliance, based on a peripheral analysis of a pulse transit time (PTT) measured at the available peripheral sites. A preliminary calibration stage extracts the arterial properties from simultaneous measurements of a pulse transit time, and an upper arm blood pressure. Obtained transfer functions, linking noninvasive peripheral measurements to the aortic pressure, cardiac output, aortic compliance and others serve to quantify the indicators of cardiac morbidity and mortality.

### CROSS REFERENCE

This application claims the benefit of the filing date of U.S. Provisional patent Application No. 62/961,819 filed Jan. 16, 2020, which is hereby incorporated by reference in its entirety.

### FIELD

The present disclosure relates to a method and wearable device for noninvasive diagnostics of proximal heart health biomarkers from peripheral measurements and a patient-specific model, and in particular to a method and wearable device for noninvasive diagnostics of proximal heart health biomarkers relating to the propagation of a pressure wave from the heart to the peripheral arteries.

### BACKGROUND

Clinical estimation of central BP, CO and AC has significant clinical implications, and is a predictor of heart disease. Historically PTT has been used to estimate upper arm BP and AC based on empirical correlations. Central BP could be estimated only invasively.

The following documents focus on predicting central cardiovascular parameters based on peripheral measurements. U.S. Pat. No. 10,398,323 to RAMAKKRISHNA MUKKAMALA, MINGWU GAO for METHOS AND APPARATUS FOR DETERMINING PULSE TRANSIT TIME AS A FUNCTION OF A BLOOD PRESSURE, issued Sep. 3, 2019, recites a “method is provided for determining pulse transit time of a subject as a function of blood pressure. The method includes: measuring a proximal waveform indicative of the arterial pulse at a proximal site of the subject; measuring a distal waveform indicative of the arterial pulse at a distal site of the subject; defining a relationship between the proximal waveform and the distal waveform in terms of unknown parameters of a nonlinear model; determining the unknown parameters of the nonlinear model from the measured proximal waveform and the measured distal waveform; and determining pulse transit time for the subject as a function of blood pressure from the parameters of the nonlinear model. The nonlinear model can account for arterial compliance and peripheral wave reflection, where the arterial compliance depends on blood pressure.” The method disclosed links the peripheral blood pressure (BP) to the central BP by a chain of interconnected elementary elements each described by the empirical exponential dependence of the compliance as a function of a BP. This method does not explore the real topology of the arterial network characterized by multiple bifurcations and multiple distal sections. The method does not utilize a physics based differential model, predicting dependence of a Pulse Transit Time (PTT) as a function of a BP, flow velocity, geometry and nonlinear physical properties of arterial branches.

U.S. Pat. No. 10,213,116 for METHODS FOR MEASURING BLOOD PRESSURE, issued Feb. 26, 2019, recites the “application relates to an apparatus and a method for estimating a central systolic blood pressure (cSBP) of a subject, in which a peripheral blood pressure waveform of the subject's pulse and at least two peripheral blood pressure measurements within the cardiac cycle of the subject are determined to provide an estimate of the central blood pressure waveform of the subject's pulse.” The method is data driven linking central BP to the peripheral one by empirical transfer function whose unknown constants are determined from the calibration procedure.

U.S. Pat. No. 9,974,450 for MEASURING CENTRAL PRESSURE WITH BRACHIAL CUFF, issued May 22, 2018, “A method for determining a calibrated aortic pressure waveform from a brachial cuff waveform involves the use of one or more generalized transfer functions. The one or more generalized transfer functions are specific for predetermined brachial cuff pressure ranges, such as below diastolic pressure, between diastolic and systolic pressure, and above systolic pressure. The brachial cuff is inflated to a pressure within the pressure range appropriate for the generalized transfer function to be applied to the brachial cuff waveform to generate the aortic pressure waveform. In some circumstances, it may be necessary to use a calibration transfer function to generate a calibrated aortic waveform. In other circumstances, the calibration transfer function is not necessary.” The method is a data driven approach linking central BP to the peripheral one by empirical transfer function whose unknown constants are determined from the calibration procedure.

U.S. Pat. No. 9,414,755 for METHOD FOR ESTIMATING A CENTRAL PRESSURE WAVEFORM OBTAINED WITH A BLOOD PRESSURE CUFF, issued Aug. 8, 2016, “A physics-based mathematical model is used to estimate central pressure waveforms from measurements of a brachial pressure waveform measured using a supra-systolic cuff. The method has been tested in numerous subjects undergoing cardiac catheterisation. Central pressure agreement was within 11 mm Hg and as good as the published non-invasive blood pressure agreement between the oscillometric device in use and the so-called “gold standard.” It also exceeds international standards for the performance of non-invasive blood pressure measurement devices. The method has a number of advantages including simplicity of application, fast calculation and accuracy of prediction. Additionally, model parameters have physical meaning and can therefore be tuned to individual subjects. Accurate estimation of central waveforms also allows continuous measurement (with intermittent calibration) using other non-invasive sensing systems including photoplethysmography.” The method uses simple linear acoustics transfer functions linking the central BP to the peripheral BP assuming linear elasticity, small deformations, absence of bifurcations.

Non-invasive blood pressure measurement could enable ambulatory or inconspicuous blood pressure monitoring. The art currently lacks an approach based on a differential physics-based model and a simple set of peripheral measurements.

### SUMMARY

In accordance with an aspect of the present disclosure, there is provided a method for noninvasive diagnostics of proximal heart health biomarkers of an individual, including:
identifying a cardiovascular sub-system, including a) a single proximal section comprising an arterial network associated with the heart, b) a plurality of distal sections and c) a plurality of cut-off sections of the identified sub-system, of the individual;
calibrating properties of the arterial network and boundary conditions by measuring a) BP at multiple locations along the sub-system, b) PTT at a location of a distal section of the plurality of distal sections, and c) a CO and arterial geometry (at least one arterial diameter) of the individual;
constructing a patient-specific model by integrating the calibrated properties into a differential physics-based fluid-structure interaction (FSI) model;
obtaining non-invasive BP and PTT diagnostic measurements in a vicinity of the location of the distal section;
running the patient-specific model in various iterations of CO until approaching the non-invasively measured BP and PTT at the distal location computing a dependency of the CO on the PTT and BP of the individual; and
deriving a proximal heart health biomarker corresponding to the distal PTT and BP diagnostic measurements.

In accordance with another aspect of the present disclosure, there is provided a device for the noninvasive diagnostics of proximal heart health biomarkers of an individual, including:
a wearable device containing at least one of a plurality of sensors (such as, PPG device, carotid artery BP measurement applanation tonometry, ECG, and ultrasound measurement of aortic diameter);
a processor; and
software containing executable code which:
   identifies a cardiovascular sub-system, comprising a) a single proximal section comprising an arterial network associated with the heart, b) a plurality of distal sections and c) a plurality of cut-off sections of the identified sub-system, of the individual;
   calibrates properties of the arterial network and boundary conditions by measuring a) BP at multiple locations along the sub-system, b) PTT at a location of a distal section of the plurality of distal sections, and c) a CO and arterial geometry (at least one arterial diameter) of the individual;
   constructs a patient-specific model by integrating the calibrated properties into a differential physics-based fluid-structure interaction (FSI) model;
   obtains non-invasive BP and PTT diagnostic measurements in a vicinity of the location of the distal section;
   runs the patient-specific model in various iterations of CO until approaching the non-invasively measured BP and PTT at the distal location computing a dependency of the CO on the PTT and BP of the individual; and
   derives a proximal heart health biomarker corresponding to the distal PTT and BP diagnostic measurements.

These and other aspects of the present disclosure will become apparent upon a review of the following detailed description and the claims appended thereto.

### BRIEF DESCRIPTION OF THE DRAWINGS

FIG. 1 is a depiction of several isolated cardiovascular subsystems in accordance with embodiments of the present disclosure;

FIG. 2 is a depiction of a right arm arterial subsystem;

FIG. 3 is a graph of an inverse reconstruction of SV and BP;

FIG. 4 is a graph showing cardiac output and upper arm blood pressure and a table showing measured data and prediction error;

FIG. 5 is a comparison of the central aortic diameter variation from doppler ultrasound and numerically reconstructed signal;

FIG. 6 is a depiction of the geometry of an upper human aorta sub-system;

FIG. 7 is an earbud-style hearable microsystem designed to provide continuous and passive cardiovascular assessment;

FIG. 8 is a flowchart showing steps for generating a patient-specific model and reconstructing CBP, CO and AC; and

FIG. 9 is a depiction of several advantages of modified CardioFAN to be utilized for central biomarker reconstruction.

### DETAILED DESCRIPTION

The disclosure relates to a method and device for the noninvasive diagnostics of proximal heart health biomarkers of an individual.

The Central Blood Pressure (CBP) is the pressure in the ascending aorta or aortic root, sending blood from the heart throughout the body. It more strongly relates to vascular disease than traditional upper arm blood pressure. It also determines the pressure in the blood vessels feeding the brain. Conventional CBP measurement procedures are invasive and can lead to complications.

The Stroke Volume (SV) is the volume of blood pumped from the heart per beat.

The Cardiac Output (CO) is the volume of blood being pumped by the heart, per unit time, CO=HR×SV.

The Pulse Wave Velocity (PWV) is the rate at which pressure waves move through a deformable vessel filled by a moving fluid. Putting this in terms of an analogy to the classical mechanics: PWV is the speed of sound waves in a compressible fluid, which is a function of the physical properties of the medium in which it travels.

The Pulse Transit Time (PTT) is the time it takes a pulse wave to travel between two arterial sites.

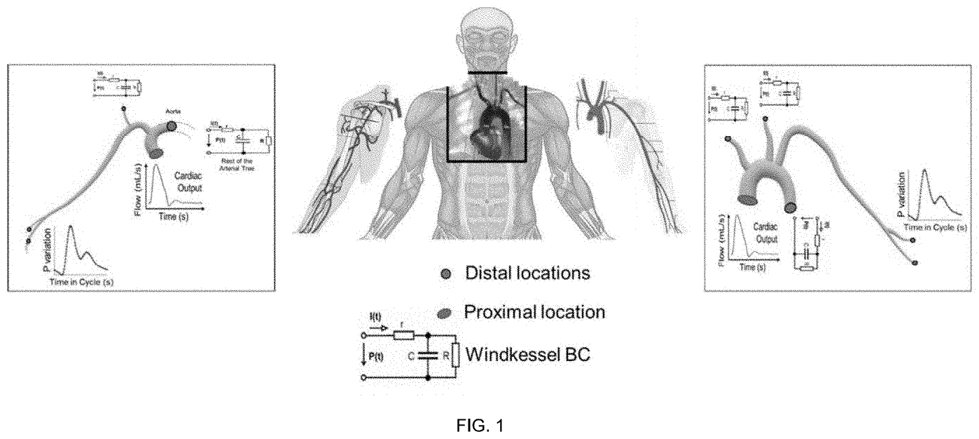

The cardiovascular system as referred to herein is a system composed of the heart, arteries, capillaries, and veins that moves blood throughout the body. A cardiovascular sub-system is a selected self-contained arterial system within the total cardiovascular system. Examples of selected sub-systems are presented in FIG. 1 which illustrates arterial sub-systems of the left and right arms.

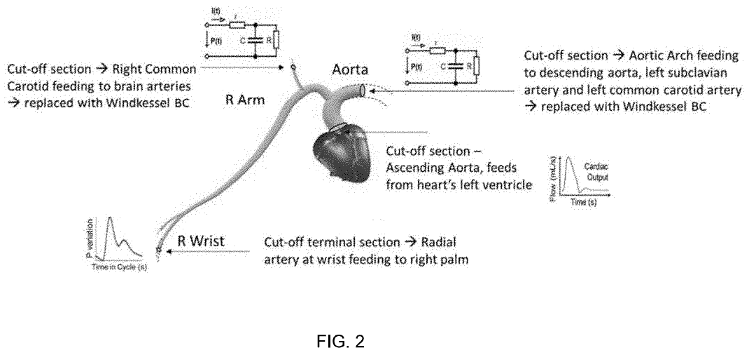

An arterial cut-off section is a virtual cut across the artery which isolates the selected arterial sub-system from the rest of the arterial system of the cardiovascular system. Examples of suitable arterial cut-off sections are presented in FIG. 2 as cut-off sections at the ascending aorta, aortic arch, carotid artery and a radial artery at the wrist.

A proximal section is a cross-section of an artery of a selected arterial sub-system nearest to the heart. The proximal section has the shortest flow path from the heart compared to other sections in a sub-system. In a preferred embodiment for estimating the CBP, the proximal section is located in the area between the aortic root and the first bifurcation in the ascending aorta. Preferably, the proximal section is the arterial cut-off section closest to the heart in the selected sub-system. For example, suitable sub-systems for estimating the CBP include: a) a sub-system starting from the aortic root to external carotid artery at the right ear (components: ascending aorta, brachiocephalic trunk, right subclavian, aortic arch, right common carotid, external and internal carotid arteries), where aortic root at the ascending aorta is the proximal section; b) a sub-system starting from aortic root to left wrist (components: ascending aorta, aortic arch, brachiocephalic, right common carotid, right subclavian, right axial, right brachial, radial and ulnar arteries), where aortic root at the ascending aorta is the proximal section; and c) a sub-system starting from the aortic root to right wrist (components: ascending aorta, brachiocephalic, right common carotid, left common carotid, descending aorta, left subclavian, left axial, left brachial, radial and ulnar arteries), where aortic root at the ascending aorta is the proximal section.

In an embodiment for estimating the BP at a location other than that for estimating the CBP, the proximal section can be located in an area other than the area between the aortic root and the first bifurcation in the ascending aorta. For example, suitable sub-systems for estimating the BP at a location other than that for the CPB include: a) a sub-system starting from the abdominal aorta to the posterior tibial artery at right or left leg, where the proximal section is a section at the abdominal aorta; and b) a sub-system starting from the left subclavian artery to the radial artery of the index finger at the left hand, where the proximal section is at the left subclavian artery.

A distal section is a cross-section of an artery of a sub-system located further away from the heart than the proximal section. FIG. 2 illustrates examples of three distal sections for the selected sub-system, which are the cut-off sections at the descending aorta, carotid artery and a radial artery at the wrist. As an additional example: in a sub-system starting from the left subclavian artery to the radial artery of the index finger at the left hand, where the distal section can be the index finger or any of the common palmar digital arteries on left hand.

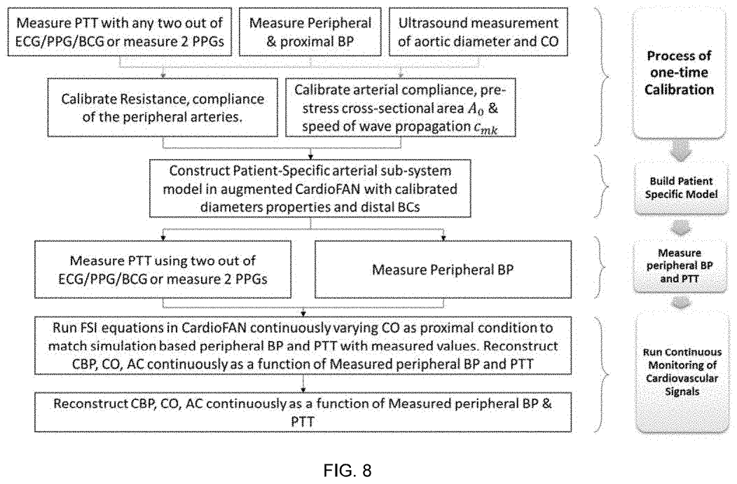

FIG. 8 is a flowchart which illustrates an embodiment of the disclosed method showing the steps of generating a patient-specific model and reconstructing CBP, CO and AC.

In accordance with an embodiment of the method, a cardiovascular sub-system is selected and isolated by identifying a single proximal section, a plurality of distal sections and a plurality of cut-off sections, as shown for example in FIG. 2.

Properties of the arterial network and boundary conditions can be calibrated by measuring local BP (other than CBP) at multiple non-invasively available measurement locations of the sub-system, for instance at the upper arm (brachial artery), neck (carotid artery), wrist, finger, and the like), PTT at a distal location, (wrist, finger, ear, and the like), and a single or plurality of arterial diameters (preferably proximal diameter) and CO of the individual.

In an embodiment, geometry of the arterial network can be measured by an imaging modality (such as ultrasound of the ascending aorta, Magnetic resonance imaging or CT scan). It can also be estimated based on statistics available on gender and height as it relates to the arterial geometry. The arterial properties, pre-stress cross-sectional lumen area, material constants and Moens-Korteweg speed of wave propagation in the absence of pressure are calibrated by measuring the aforementioned parameters to satisfy equations described in the following steps.

Calibration procedure: an isolated cardiovascular sub-system is identified with a single proximal section associated with the heart, and a number of distal sections, one of which is located at the PTT point of measurement (left or right wrist, finger, leg, temple, buttocks, etc.), while the remainder of the distal sections relate to the artificial cut-off sections. Examples of the two isolated cardiovascular sub-systems, pertaining to right and left hands are presented in FIG. 1

FIG. 1 illustrates examples of isolated cardiovascular subsystems, from heart to right arm (left picture) and from heart to left arm (right picture). Distal cross sections are replaced with a Windkessel boundary condition to account for the resistance and compliance of the rest of the truncated arteries. The distal section that is replaced with pressure measurement from the wrist is at the radial and ulnar arteries at the wrist. In these sub-systems the proximal section is the closest cross-section with lowest flow path to heart's left ventricle. Since in this sub-system it is directly connected to the left ventricle (without any bifurcations), the cardiac output can be associated to this section.

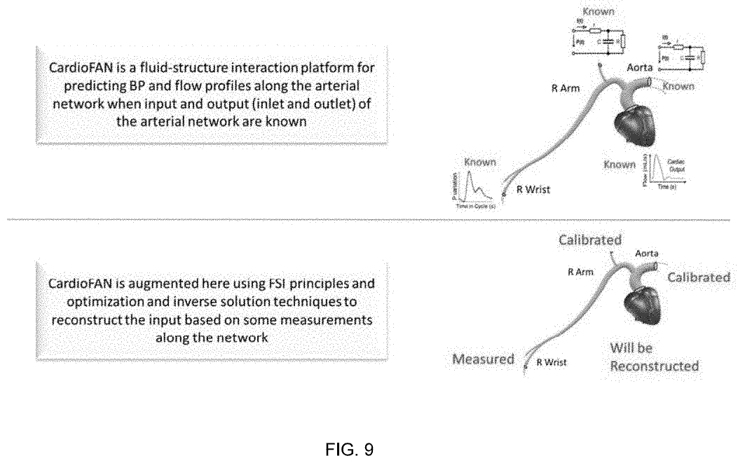

A patient specific calibration procedure is conducted for each patient, with an objective to identify geometric and material constants, as well as parameters of the Windkessel boundary conditions. FIG. 9 shows advantages of a modified CardioFAN utilized for central biomarker reconstruction.

The Windkessel boundary conditions are based on an electrical analogy where an arterial tree is assimilated to an electric circuit. The parameters of the components of the circuit (resistances, capacitances, etc.) correspond to the properties of each branch: the arterial compliance is represented as a capacitor with electric charge storage properties; peripheral resistance of the systemic arterial system is represented as an energy dissipating resistor. FIG. 2 shows a right arm arterial subsystem representing the Windkessel conditions at the cut-off sections at the carotid artery and a descendent aorta. The other two cut-off sections shown in FIG. 2 are characterized by the cardiac output (ascending aorta) and the blood pressure waveform at radial artery at the wrist.

Calibration of geometry and material constants: The nonlinear hyperelastic deformation of a biological tissue is described by the Y. C. Fung model, or the mathematical model of Holzapfel-Gasser, both express as anisotropic super-elastic properties. The specific potential energy U_{c} according to Fung is delineated by the following equation (1), where c and a₁₁ are material constants, η—circumferential strain, A, A₀—arterial cross-sectional areas in the presence of intramural pressure P, and a pressure free condition respectfully.

$${U}_{e} = \frac{c}{2} ( {e}^{{a}_{11} {η}^{2}} - 1 ) , η = \sqrt{\frac{A}{{A}_{0}}} - 1 \qquad ( 1 )$$

Equation (1) can be transformed to the “physics based” tube law as presented by Eq. (2) (Y. Seyed Vahedein and A. S. Liberson, “CardioFAN: Open source platform for noninvasive assessment of pulse transit time and pulsatile flow in hyper-elastic vascular networks”, en, Biomechanics and Modeling in Mechanobiology, May 2019) which is incorporated herein by reference in its entirety.

P=2ρ_{f}cₘₖ²ηe^{a}^{₁₁}^{η}^{²}

where ρ_{f}—is the blood density, cₘₖ—is the Moens-Korteweg speed of wave propagation in the absence of pressure. The vector of geometric-mechanical properties \vec{V₁}=(A₀, cₘₖ, a₁₁) requires preliminary evaluation (calibration procedure).

Calibration of parameters of the Windkessel boundary conditions, applied to the distal cut-off sections:

Windkessel parameters can be identified for designing a patient-specific model (FIG. 2).
The typical 3 elements Windkessel model is found to be

$$( 1 + \frac{{R}_{1}}{{R}_{2}} ) Q ( t ) + C {R}_{1} \frac{dQ ( t )}{dt} = \frac{P ( t )}{{R}_{2}} + C \frac{dQ ( t )}{dt} \qquad ( 3 )$$

where R₁, R₂—resistances, C—capacitance, Q—flow rate. The vector of unknown parameters \vec{V₂}=(R₁, R₂, C) is specified based on a calibration procedure. The variety of Windkessel models could be found in a number of sources, for instance in Solving Windkessel Models with MLAB (civilized.com).

During the calibration procedure the measurements can be recorded in supine and sitting positions and include: wrist BP, brachial BP, cardiac output, and the PTT from heart to wrist. The PTT from heart to wrist can be measured using any combination of the following: a) wrist photoplethysmography (PPG), and electro-cardiography (ECG); b) two PPGs, one at a proximal artery and another at wrist; c) wrist PPG and balistocardiogram (BCG). The various PTT measurement techniques are reviewed in (Ding X, Zhang Y-T, “Pulse transit time technique for cuffless unobtrusive blood pressure measurement: from theory to algorithm” en Biomedical Engineering Letters, 2019). As a result we have the following measured quantities: SBP_{arm,meas}ⁱ—systolic upper arm blood pressure at the supine (i=1) and sitting (i=2) positions;

DBP_{arm,meas}ⁱ—diastolic upper arm blood pressure at the supine (i=1) and sitting (i=2) positions;
PTTₘₑₐₛⁱ—pulse transit time required for pulse to travel from the heart to the wrist at the supine (i=1) and sitting (i=2) positions.
D_{sys,meas}ⁱ—systolic aortic diameter at the supine (i=1) and sitting (i=2) positions;
D_{dias,meas}ⁱ—diastolic aortic diameter at the supine (i=1) and sitting (i=2) positions.

The numerical solution of the reduced 1D model (Y. Seyed Vahedein and A. S. Liberson, “CardioFAN: Open source platform for noninvasive assessment of pulse transit time and pulsatile flow in hyper-elastic vascular networks”, en, Biomechanics and Modeling in Mechanobiology, May 2019) is a solution of a relating system of partial differential equations satisfying the measured amount of a stroke value as a boundary condition at the inlet of the cardiovascular subsystem, and the measured SBP and DBP pressure at the wrist. The distal cut-off sections are characterized by the Windkessel conditions as it was described in [0032]. As a result, the measured quantities can be presented as the functions of calibrated parameters \vec{V₁}, \vec{V₂}, i.e. SBP_{arm,cal}ⁱ(\vec{V₁}, \vec{V₂}), DBP_{arm,cal}ⁱ(\vec{V₁}, \vec{V₂}), PTT_{cal}ⁱ(\vec{V₁}, \vec{V₂}), D_{sys,cal}ⁱ(\vec{V₁}, \vec{V₂}), D_{dias,cal}ⁱ(\vec{V₁}, \vec{V₂}).

The above properties can be determined requesting identity of the measured and calculated quantities

$$R E {S}_{1}^{i} ( \vec{{V}_{1}} , \vec{{V}_{2}} ) = \frac{S B {P}_{a r m , c a l}^{i} ( \vec{{V}_{1}} , \vec{{V}_{2}} )}{S B {P}_{a r m , m e a s}^{i}} - 1 = 0 \\ R E {S}_{2}^{i} ( \vec{{V}_{1}} , \vec{{V}_{2}} ) = \frac{D B {P}_{a r m , c a l}^{i} ( \vec{{V}_{1}} , \vec{{V}_{2}} )}{D B {P}_{a r m , m e a s}^{i}} - 1 = 0 \\ R E {S}_{3}^{i} ( \vec{{V}_{1}} , \vec{{V}_{2}} ) = \frac{P T {T}_{c a 1}^{i} ( \vec{{V}_{1}} , \vec{{V}_{2}} )}{P T {T}_{m e a s}^{i}} - 1 = 0 \\ R E {S}_{4}^{i} ( \vec{{V}_{1}} , \vec{{V}_{2}} ) = \frac{{D}_{s y s , c a l}^{i} ( \vec{{V}_{1}} , \vec{{V}_{2}} )}{{D}_{s y s , m e a s}^{i}} - 1 = 0 \\ R E {S}_{5}^{i} ( \vec{{V}_{1}} , \vec{{V}_{2}} ) = \frac{{D}_{d i a s , c a l}^{i} ( \vec{{V}_{1}} , \vec{{V}_{2}} )}{{D}_{d i a s , m e a s}^{i}} \qquad ( 4 )$$

which could be achieved, for instance, by a direct minimization procedure

Σᵢ₌₁Σₖ₌₁⁵(RESₖⁱ(\vec{V₁},\vec{V₂}))²→MIN (5)

Once a calibration procedure is performed, non-invasive peripheral measurements of a BP and a PTT are taken of the individual. The non-invasive diagnostics of proximal heart health biomarkers: aortic (CBP), stroke volume (SV) and aortic compliance (AC) are calculated, using peripheral measurements of a BP and a PTT. The method enables calculation of the central BP, CO and AC from the non-invasive peripheral BP and PTT measurements from any peripheral site, for example from the wrist, as shown in FIG. 1 using a high accuracy physics based differential fluid-structure interaction (FSI) model.

Arterial compliance (AC) is defined as the ratio of change of volume to change in pressure (or the slope of the pressure volume curve), serving as a predictable marker for vascular disease states.

Photoplethysmography (PPG) is a simple low-cost optical technique that can be used to detect blood volume changes in the microvascular bed of tissue. Pulse transit time (PTT) is often derived from calculations on ECG and PPG signals and is based on tightly defined characteristics of the waveform.

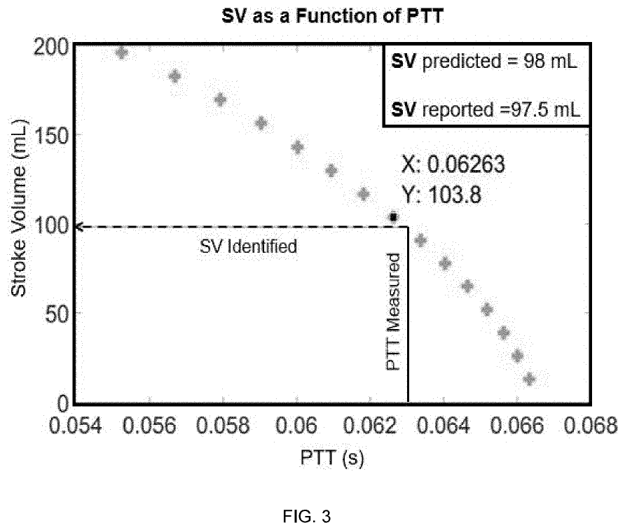

Specifying a set of physiologically relevant ranges of stroke volume values (SVᵢ), the model is assigned with the specified SVᵢ and the measured peripheral BP as boundary conditions at the inlet and the terminal distal section of the cardiovascular subsystem respectfully. For each model run, calculate the corresponding PTTᵢ, so that the digital function PTTᵢ=PTT(SVᵢ) is obtained (green markers in FIG. 3 Inverse Reconstruction of SV and BP), thereby at each run the PTT is calculated as a function of SV. Cardiac output is related to the stroke volume with the following relationship: CO=SV*Heart Rate (HR).

An important feature of the disclosure is the insight on a dependence of proximal heart health biomarkers, such as central blood pressure (CBP), cardiac output (CO), stroke volume (SV) and aortic compliance (AC) on a peripheral blood pressure waveform and a pulse transit time (PTT), relating to the propagation of a pressure wave from the heart to the peripheral arteries. The latter allows to diagnose noninvasively the proximal biomarkers by peripheral measurements of a BP and a PTT coupled with the calibrated physics-based differential model.

The method and device can be used to provide an accurate noninvasive estimate of central BP, cardiac output and aortic compliance, which are of high clinical significance. An immediate advantage of the method is in diagnosis and management of hypertension. As it is demonstrated in prior art [Cheng H-M, Chuang S-Y, Wang T-D, et al., “Central blood pressure for the management of hypertension: Is it a practical clinical tool in current practice?” En The Journal of Clinical Hypertension 2020], the CBP is a far more accurate indicator of early signs of hypertension. It is the closest blood pressure measurement to the left ventricle of the heart and captures the changes of pressure at the heart compared to a more distal location. In addition, treatment of hypertension and guidance of BP using medication can be more efficient using CBP. Thereby, introduction of a noninvasive and accurate CBP measurement technique would help in measuring these effects more efficiently.

Other potential uses for this method include: 1) measurement of CBP for detecting coronary artery disease. The CBP is typically estimated when performing a computed tomography angiography (CTA)-derived fractional flow reserve (FFR) technique to monitor the blockage of coronary arteries. The method presented here can help more accurately provide an aortic BP; 2) measurement of CBP for detecting the effect of arterial stents on aortic BP before and after the procedure; 3) study the effect of different drugs on CBP and pressure in heart ventricles.

An embodiment of the measurement setup provides estimation of a peripheral blood pressure and a pulse transit time, which are converted using the patient specific computational model to the set of central aortic cardiovascular markers. The latter has a potential to build the feasible foundation for the personalized continuous self-monitoring of cardiovascular health based on portable mobile and wearable applications. An accurate and reliable pulse transit time based central blood pressure estimation is achieved despite the complexity of BP regulation in the human body. The method provides an accurate approach using a physics-based differential model and a simple set of peripheral measurements.

A preferred configuration uses a peripheral blood pressure measurement (arm or wrist) in combination with a peripheral pulse transit time (PTT) to estimate central cardiac parameters including central aortic blood pressure, cardiac output, and aortic compliance. A benefit of this technology is that it can be used to monitor these central characteristics with inexpensive technology in the clinic or the home. The benefits go well beyond what is available with an arm blood pressure cuff and have greater clinical significance.

The disclosure differs from prior technology in that it uses a physics-based calibrated differential model, estimating accurately cardiac biomarkers based on simple peripheral measurements of PTT and BP.

Features of the present method include: calibration of the human arterial network based on measurements of brachial and wrist BPs, PTT, CO; predicting PTT based on a physics based model; once calibration is completed, predicting CO based on measured PTT; it allows noninvasive measurements of a central BP, which can be presently measured only invasively.

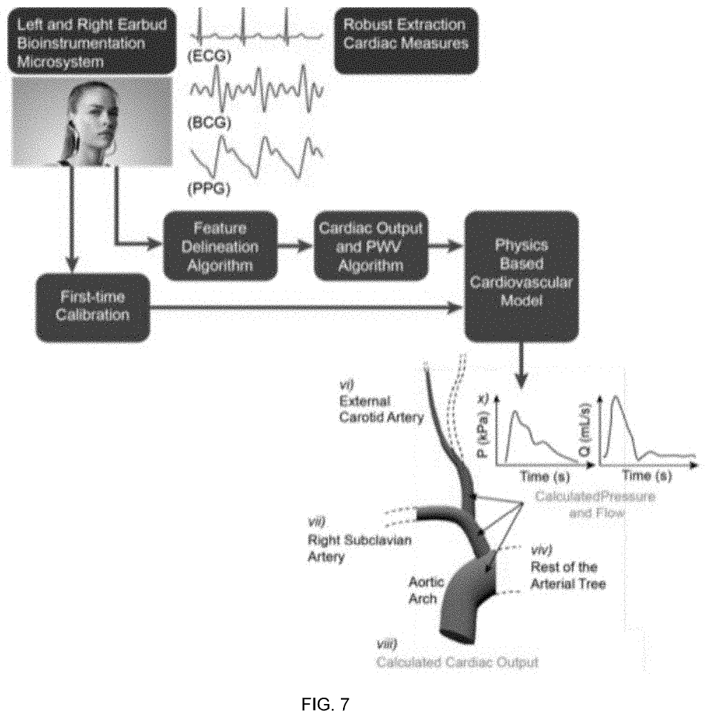

A wearable device can be a watch, glasses, earbuds, shoes, etc., containing at least one of a plurality of sensors in a system on a chip (SoC) PCBA board, including a PPG sensor, carotid artery BP measurement applanation tonometry, two-lead ECG, Force Sensitive Resistors (FSR) to measure Balistocardiogram (BCG) and ultrasound measurement of aortic diameter; a processor; memory; and software containing executable code which performs the present method. The present method implements a high accuracy differential model to wearable devices, enabling continuous noninvasive monitoring of cardiovascular health to facilitate cardiovascular diagnostics at early stages of cardiovascular comorbidities. This algorithm can be embedded into the memory of a wearable device that is capable of measuring any two combination of PPG/ECG/BCG (such as apple-watch series 6) firmware. The software will utilize ECG and PPG information from wearable device sensors to extract PTT and additionally will take BP measurement at wrist (either in a wearable that already has a way to measure BP such as Omron Heartguide or using a wrist cuff BP monitoring device) to calculate CBP, CO and AC. Analysis can be performed on wearable processor and the data stored on the memory. It is envisioned that the wearable being an internet of things (IoT) device, it can store the cardiovascular signal data wirelessly on a server or a hub computer. This approach can both quantify and visually depict distributions of a cardiac output, blood pressure inside the central aorta and carotid artery, along with other important cardiac measures. The systems procedure is composed of three distinct stages: (1) Data acquisition; (2) Cardiovascular model calibration (personification); (3) Assessment of cardiovascular markers. In another example wearable, the peripheral PPG and BCG data is extracted using earbud bioinstrumentation microsystem. Systolic and diastolic BP are measured at the carotid artery. For the purpose of calibration ECG, cardiac output (CO) and ultrasound measurements are performed once at the aortic arch. The system will be able to quantify and present important cardiovascular measures such as distributions of heart rate (HR), heart rate variability (HRV), electrocardiogram (ECG), pre-ejection period (PEP), cardiac output (CO), aortic blood pressure (BP), and arterial compliance along the aorta, all based on a peripheral measurement of PPG and BCG at the ears (FIG. 7). FIG. 7 shows an earbud-style hearable microsystem designed to provide continuous and passive cardiovascular assessment. Key cardiovascular waveforms are captured by the hearable device, custom algorithms extract key features and calculate cardiac parameters of PWV and CO, with the model calculating both aortic and peripheral pressures and flow at different points in the vasculature.

The disclosure will be further illustrated with reference to the following specific examples. It is understood that these examples are given by way of illustration and are not meant to limit the disclosure or the claims to follow.

### EXAMPLES

The bio-instrumental system was tested for three evaluated cases. Reconstructed results show average standard deviation of 1.4% for systolic and 4.6% for diastolic BPs, as well as 8.4% for cardiac output. Results show maximal across the aorta deviation of the flow rate as 4.1% and cross section area as 2.7% from the clinically measured values. Prediction of a stroke volume was within 0.5% of the measured value (from MRI).

### Example 1

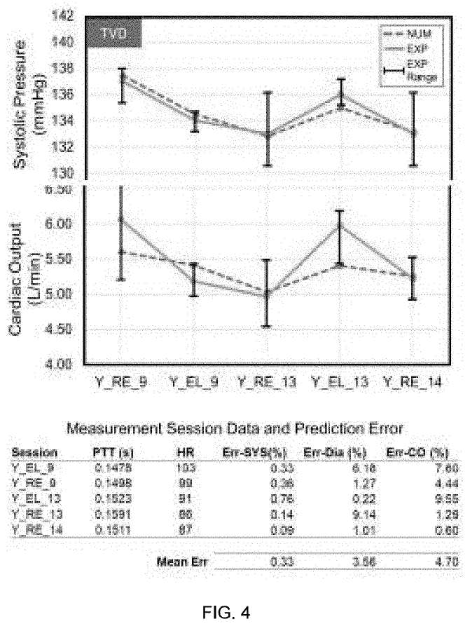

The human subject is a 33-year old male. The PTT, CO and BP measurements have been conducted using peripheral measurements at the wrist, as depicted in the FIG. 3. The calibration and validation procedures have been conducted following the methodology described above. FIG. 4 shows the BP and cardiac output predictions as a function of different recording sessions. Parameters predicted using the method presented in this patent are compared with the corresponding values measured using the gold standard measurement techniques and individual errors as well as the mean prediction errors are reported as a table in FIG. 4.

FIG. 4 shows cardiac output and upper arm blood pressure. Inversely reconstructed values (dotted blue line) obtained using TVD version of the created software, entitled CardioFAN, against experimental measurements (solid orange line). The attached table displays the error of a CardioFAN prediction vs set of measurements serving for validation. EL: elevated HR, RE: resting HR, CO: cardiac output.

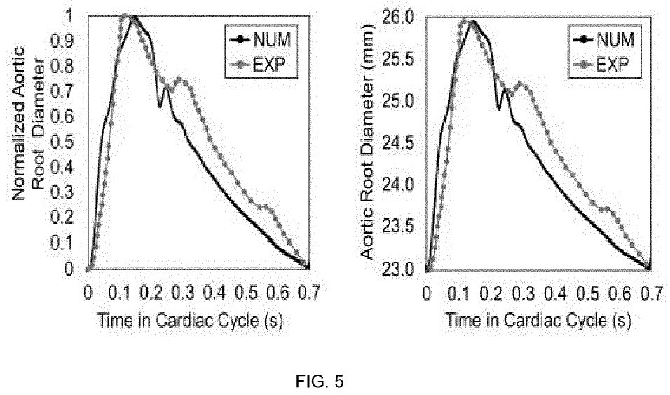

FIG. 5 is a comparison of the central aortic diameter variation from doppler ultrasound and numerically reconstructed signal. Normalized (left) and absolute (right) variations are demonstrated. The central diameter variation reported by CardioFAN is compared against the shape of diameter changes in one cardiac cycle, measured by doppler ultrasound. Normalized and absolute values are compared at the ascending aorta, showing very promising results.

### Example 2

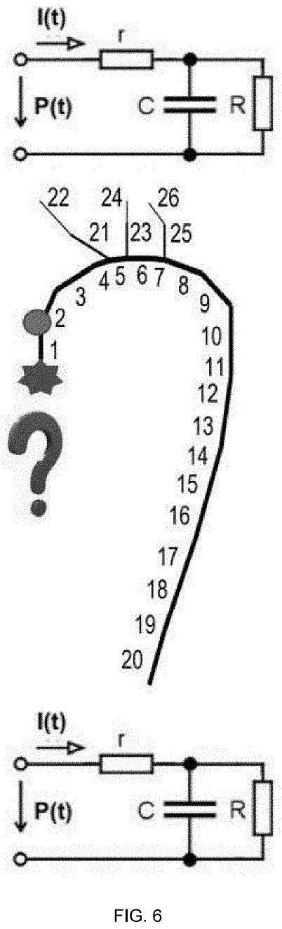

In FIG. 6 geometry of the upper human aorta sub-system is identified, showing its 26 segments. Each segment in the sub-system is numbered and shown along the subsystem and they are referring to various arterial geometry captured inside the subsystem. The terminal sections are closed with the Windkessel BCs. An unknown cardiac output is identified by the question mark and is calculated using the present method.

The aortic geometry is represented by interconnected 20 segments (FIG. 6) each modeled as a deformable vessel with properties depending on a single axial coordinate. The input data—geometric and mechanical properties, the Windkessel model and the peripheral measurements taken at the segment 19 have been drawn from the paper J. Alastruey, N. Xiao, H. Fok, T. Schaeffter, and C. A. Figueroa, “On the impact of modelling assumptions in multi-scale, subject-specific models of aortic haemodynamics”, en, Journal of The Royal Society Interface, vol. 13, no. 119, p. 20, Jun. 2016. Specifying a set of plausible stroke volumes at the inlet (segment 2, FIG. 6). and the measured peripheral conditions at the distal segment 19 (FIG. 6) the digital function PTTᵢ=PTT(SVᵢ) is obtained, as visualized by markers shown in FIG. 3. The value of a stroke volume predicted by the described method is 98.0 ml vs 97.5 ml obtained by clinical measurements. The simulated systolic and diastolic pressure in a left carotid artery (segment 24, FIG. 6) are within 2% of the clinically measured quantities. The relating predicted volume flow rate is 285 ml/s vs 270 ml/s the measured value, which corresponds to the 5.5% of error.

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

What is claimed:

1. A method for noninvasive diagnostics of proximal heart health biomarkers of an individual, comprising:
identifying a cardiovascular sub-system, comprising a) a single proximal section comprising an arterial network associated with the heart, b) a plurality of distal sections and c) a plurality of cut-off sections of the identified sub-system, of the individual;
calibrating properties of the arterial network and boundary conditions by measuring a) BP at multiple locations along the sub-system, b) PTT at a location of a distal section of the plurality of distal sections, and c) a CO and an arterial diameter of the individual;
constructing a patient-specific model by integrating the calibrated properties into a differential physics-based fluid-structure interaction (FSI) model;
obtaining non-invasive BP and PTT diagnostic measurements in a vicinity of the location of the distal section;
running the patient-specific model in various iterations of CO until approaching the non-invasively measured BP and PTT at the distal location computing a dependency of the CO on the PTT and BP of the individual; and
deriving a proximal heart health biomarker corresponding to the distal PTT and BP diagnostic measurements.

2. The method of claim 1, further comprising continuously deriving the proximal heart health biomarker by continuous non-invasive measuring of the distal BP and PTT.

3. The method of claim 1, wherein the proximal heart health biomarker includes at least one of central blood pressure (CBP), cardiac output (CO), stroke volume (SV) and aortic compliance (AC).

4. The method of claim 1, wherein the model uses principles of fluid and structure interaction to generate a calibrated patient-specific arterial sub-system that can continuously reconstruct cardiovascular health biomarkers.

5. The method of claim 1, wherein the calibrated properties of the boundary conditions include Windkessel properties of resistance and compliance of truncated arteries.

6. The method of claim 1, wherein the calibrated properties of the arterial network include arterial compliance, speed of wave propagation and cross-sectional area of arterial wall in absence of pressure.

7. A device for the noninvasive diagnostics of proximal heart health biomarkers of an individual, comprising:
a wearable device containing at least one of a plurality of sensors;
a processor; and
software containing executable code which:
identifies a cardiovascular sub-system, comprising a) a single proximal section comprising an arterial network associated with the heart, b) a plurality of distal sections and c) a plurality of cut-off sections of the identified sub-system, of the individual;
calibrates properties of the arterial network and boundary conditions by measuring a) BP at multiple locations along the sub-system, b) PTT at a location of a distal section of the plurality of distal sections, and c) a CO and arterial geometry of the individual;
constructs a patient-specific model by integrating the calibrated properties into a differential physics-based fluid-structure interaction (FSI) model;
obtains non-invasive BP and PTT diagnostic measurements in a vicinity of the location of the distal section;
runs the patient-specific model in various iterations of CO until approaching the non-invasively measured BP and PTT at the distal location computing a dependency of the CO on the PTT and BP of the individual; and
derives a proximal heart health biomarker corresponding to the distal PTT and BP diagnostic measurements.

8. The device of claim 7, wherein the sensor includes at least one of an PPG device, carotid artery BP measurement applanation tonometry, ECG, and ultrasound measurement of aortic diameter.
